(19) United States
(12) Patent Application Publication
(10) Pub. No.: US 2011/0135607 A1
(43) Pub. Date: Jun. 9, 2011

(54) VIRAL NUCLEIC ACID FOR THE TREATMENT OF NEURODEGENERATIVE DISORDERS

(76) Inventors: John Sinclair, Cambridge (GB); Matthew Reeves, Cambridge, MA (US)

(21) Appl. No.: 12/994,327

(22) PCT Filed: May 22, 2009
(86) PCT No.: PCT/GB2009/001293

§ 371 Date: Nov. 23, 2010

(30) Foreign Application Priority Data
May 23, 2008 (GB) 0809476.5

(51) Int. Cl. A61K 35/66; C07H 21/02; C12N 15/63; C12N 1/21; C07K 9/00; A61K 31/7088; A61K 38/14; A61P 25/16; A61P 25/28; C12P 19/34; C12N 5/10; C12N 1/19

(52) U.S. Cl. 424/93.2; 536/23.1; 435/320.1; 435/252.33; 530/322; 514/44 R; 514/17.7; 514/17.8; 435/91.1; 435/325; 435/254.2

(57) ABSTRACT

This invention relates to a non-coding RNA (ncRNA) sequence from Human cytomegalovirus (HCMV) (termed “TRL4”) that specifically protects neuronal cells from cell death and may therefore be useful in the treatment of neurodegenerative disorders, such as Parkinson's disease.

### FIELD OF INVENTION

This invention relates to the prevention of neuronal cell death and provides agents and methods which may be useful, for example, in the prevention of neurodegeneration and the treatment of neurodegenerative disorders.

### BACKGROUND OF INVENTION

Parkinson's disease (PD) is a common neurodegenerative disorder of the CNS in which there is selective loss of populations of neurons, especially the dopaminergic cells of the substantia nigra. The pathogenesis of sporadic PD is unclear but abnormalities in complex I of mitochondria have been well described (Schapira et al., 1990; Pyle et al., 2004), the relevance of which has recently been reinforced by the identification of the genes underlying certain rare familial forms of PD. In some of these familial forms of PD, whilst there is no evidence for a direct role for complex 1 abnormalities, those involving mutations in PINK 1, DJ1, parkin and alpha-synuclein are also often associated with mitochondrial dysfunction and oxidative stress. Specific targeting of the mitochondrial Complex I with the pesticide rotenone produces degeneration of dopaminergic neurons in many primary cell culture models and, in vivo, it also causes motor dysfunction.

Human cytomegalovirus (HCMV) expresses a non-coding RNA (ncRNA) transcript (McSharry et al 2003). Expression of the ncRNA during human cytomegalovirus (HCMV) infection has been shown to prevent cell death during the extended time course of infection that occurs with this ubiquitous human herpesvirus (Reeves et al. 2007).

### SUMMARY OF INVENTION

The present inventors have identified a non-coding RNA (ncRNA) sequence (termed “TRL4” herein) that specifically protects neuronal cells from cell death in cell-based and animal models of neurodegenerative disorders and may therefore be useful in the development of therapies to treat such disorders.

An aspect of the invention provides a TRL4 nucleic acid comprising a nucleotide sequence having at least 60% sequence identity to SEQ ID NO: 1.

Another aspect of the invention provides a TRL4 nucleic acid for use in a method of treatment of the human or animal body.

Another aspect of the invention provides a method of treatment of a neurodegenerative disorder in individual in need thereof comprising;
administering a TRL4 nucleic acid to the individual.

Another aspect of the invention provides a TRL4 nucleic acid for use in a method of treating a neurodegenerative disorder.

Another aspect of the invention provides the use of a TRL4 nucleic acid for use in the manufacture of a medicament for use in a method of treating a neurodegenerative disorder.

### BRIEF DESCRIPTION OF DRAWINGS

FIG. 1 show phase contrast micrographs and FITC staining of non-neuronal HEK293 and neuronal U373 cells treated with TRL4 RNA and RVG peptide.

FIG. 2 shows Hoeschst staining and TUNEL fluorescence of SH-SY5Y neuronal cells treated with TRL4 RNA and RVG peptide and subsequently treated with rotenone.

FIG. 3 shows the relative proportions of TH positive cells in rat 13.5 day foetal ventral midbrain primary cultures pretreated with TRL4(ncRNA) or control actin (actin), followed by treatment with 60 uM 6-OHDA for 48 hours and staining for tyrosine hydroylase.

FIG. 4 shows the experimental protocol for assessing the neuroprotective effect of the TRL4/peptide complex in the 6-OHDA rat model.

FIG. 5 shows the results of TH staining of rat brain sections after direct injection of the TRL4/peptide complex into the substantia nigra, showing that TRL4/peptide complex treated rats but not control actin/peptide complex treated rats are protected from the 6-OHDA lesion.

FIG. 6 shows the results of behavioural analysis which indicates that TRL4/peptide complex treated rats but not control actin/peptide complex treated rats are protected from behavioural impairments associated with 6-OHDA lesions.

### DETAILED DESCRIPTION OF THE INVENTION

The present invention relates to the TRL4 nucleic acids and the use of TRL4 nucleic acids in the treatment of medical or veterinary conditions, including neurodegenerative conditions.

A TRL4 nucleic acid may comprise the nucleotide sequence of SEQ ID NO: 1 or a variant of SEQ ID NO: 1 or a fragment of such a nucleotide sequence.

A nucleotide sequence which is a variant of a reference TRL4 nucleotide sequence set out herein, such as SEQ ID NO: 1, may share at least 50% sequence identity with the reference nucleotide sequence, preferably at least 60%, at least 70%, at least 80%, at least 90%, at least 95% or at least 98% sequence identity.

Particular nucleotide sequence variants may differ from the reference nucleotide sequence, such as SEQ ID NO: 1 by addition, deletion or substitution of 1 or more nucleotides, for example, up to 5 nucleotides, up to 10 nucleotides, up to 20 nucleotides, up to 30 nucleotides, up to 40 nucleotides, up to 50 nucleotides, up to 60 nucleotides, up to 70 nucleotides, up to 100 nucleotides, up to 150 nucleotides, up to 200 nucleotides or up to 250 nucleotides.

Sequence identity is commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Sequence comparison may be made over the full-length of the relevant sequence described herein.

In other embodiments, a nucleotide sequence which is a variant of a reference nucleotide sequence, such as SEQ ID NO: 1 may selectively hybridise under stringent conditions with the reference nucleotide sequence or the complement thereof.

Stringent conditions include, e.g. for hybridization of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

Variants of SEQ ID NO: 1 may include sequences corresponding to SEQ ID NO: 1 from other HCMV isolates. These sequences may be identified using standard sequence analysis tools. Examples of variant sequences include the nucleotide sequences of NCBI database identifiers: AY325310.1 GI: 33114658 (nucleotides 1 to 793; SEQ ID NO: 5); AY325311.1 GI: 33114659 (nucleotides 1 to 799; SEQ ID NO: 6), AY325312.1 GI: 33114660 (nucleotides 1 to 795; SEQ ID NO: 7) and X17403.1 GI: 59591 (reverse complement of nucleotides 3782 to 4575; SEQ ID NO: 8) (McSharry et al 2003).

A variant TRL4 nucleotide sequence preferably retains one or more functional characteristics of SEQ ID NO: 1, for example the ability to protect neuronal cells from rotenone-induced cell death.

A fragment of a full-length sequence may be at least 400, at least 500, at least 600 or at least 700 contiguous nucleotides of SEQ ID NO: 1 or a variant thereof. Preferably, a fragment retains one or more functional characteristics of SEQ ID NO: 1. For example, a TRL4 nucleic acid may comprise the nucleotide sequence of nucleotides 1 to 281 or nucleotides 282-795 of SEQ ID NO: 1 or a variant thereof.

In some embodiments, a suitable fragment may comprise a nucleotide sequence having at least 60%, at least 70%, at least 80%, at least 90% or at least 95% sequence identity to the sequence of nucleotides 1 to 281 or nucleotides 282-795 of SEQ ID NO: 1.

SEQ ID NO: 1 corresponds to nucleotides 1-795 of SEQ ID NO: 2. In some embodiments, a TRL4 nucleic acid may comprise the nucleotide sequence of SEQ ID NO: 2 or a variant thereof. In other embodiments, a TRL4 nucleic acid may consist of a fragment, for example at least 400, at least 500, at least 600 or at least 700 contiguous nucleotides and up to 1000, up to 1500 or up to 2000 contiguous nucleotides of SEQ ID NO: 2 or a variant thereof. Preferably, a fragment retains one or more functional characteristics of the TRL4 sequences described herein. In some embodiments, a suitable fragment may comprise nucleotides 282-795 of SEQ ID NO: 2, preferably residues 1 to 795 of SEQ ID NO: 2.

Variant nucleotide sequences are described in more detail above. Examples of variants of SEQ ID NO: 2 include the nucleotide sequences of NCBI database entries AY325310.1 GI: 33114658; AY325311.1 GI: 33114659; and AY325312.1 GI: 33114660.

Optionally, heterologous nucleotide sequences may be attached to the 5′ and/or 3′ termini of the TRL4 nucleic acid. For example, a heterologous nucleotide sequence having up to 5, up to 10, up to 100, up to 500 or up to 1000 nucleotides may be attached to the TRL4 nucleic acid. An isolated nucleic acid may comprise a TRL4 nucleic acid as described herein and one or more heterologous nucleotide sequences.

A heterologous nucleotide sequence is a nucleotide sequence which is not naturally associated with the TRL4 nucleic acid in the HCMV viral sequence. For example, a heterologous nucleotide sequence may be an artificial sequence (i.e. not found in nature), a non-viral nucleotide sequence or a non-HCMV nucleotide sequence.

The TRL4 nucleic acid may be a DNA molecule, an RNA molecule or a modified DNA or RNA molecule.

TRL4 nucleic acid as described herein may be readily prepared by the skilled person using the information and references contained herein and techniques known in the art (for example, see Molecular Cloning: a Laboratory Manual: 3rd edition, Sambrook and Russell (2001) Cold Spring Harbor Laboratory Press). For example, TRL4 nucleic acid may be prepared by conventional solid phase synthesis techniques or may be produced by recombinant means. In preferred embodiments, a TRL4 RNA molecule may be prepared by in vitro transcription from a template TRL4 nucleic acid using standard techniques.

A TRL4 nucleic acid, for example a TRL4 DNA or RNA molecule, may comprise one or more modifications. For example, a TRL4 nucleic acid may comprise a modified polynucleotide backbone; modifications to one or more bases; and/or modifications to one or more sugar moieties.

A modified nucleic acid backbone may comprise one or more non-natural internucleoside linkages. A modified backbone may include phosphorus atoms or may lack phosphorus atoms.

Examples of modified nucleic acid backbones include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Examples of modified nucleic acid backbones lacking phosphorus atoms include backbones formed by short chain alkyl or cycloalkyl internucleoside linkages, mixed heteroatom and alkyl or cycloalkyl internucleoside linkages, or one or more short chain heteroatomic or heterocyclic internucleoside linkages. These include backbones having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other examples of modified nucleic acid backbones, both the sugar and the internucleoside linkage, i.e. the backbone, of the nucleotide units are replaced with non-naturally occurring groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

A TRL4 nucleic acid may comprise one or more substituted sugar moieties or may contain one or more sugar mimetics such as cyclobutyl moieties in place of the pentofuranosyl sugar.

A TRL4 nucleic acid may comprise one or more base modifications or substitutions. For example a natural base, such as adenine (A), guanine (G), thymine (T), cytosine (C) or uracil (U) may be replaced by a modified base. Modified bases include other synthetic and natural bases such as 5-methylcytosine (5-Me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

A TRL4 nucleic acid comprising one or more modifications may be synthesised using standard solid phase techniques.

In some embodiments, terminal protecting groups may be attached to the 5′ and/or 3′ termini of the TRL4 nucleic acid or an isolated nucleic acid encoding the TRL4 nucleic acid. Terminal protecting groups may protect the TRL4 nucleic acid from degradation in vivo. Suitable protecting groups are well-known in the art (e.g., Greene e al., (1991) Protective Groups in Organic Synthesis, 2nd ed., John Wiley & Sons, Inc. Somerset, N.J.) and include acetyl, amide, and 3 to 20 carbon alkyl groups.

In some embodiments, a TRL4 nucleic acid as described herein may be provided isolated and/or purified e.g. in substantially pure or homogeneous form. Isolated TRL4 nucleic acid will be free or substantially free of material, such as proteins and nucleic acids, with which it is found in its natural environment (i.e. the human cytomegalovirus), or the environment in which it is prepared (e.g. in vitro transcription reactions). Preferred isolated TRL4 nucleic acids include TRL RNA molecules.

TRL4 nucleic acid may be contained within a vector or host cell and/or formulated with diluents or adjuvants and still be isolated—for example TRL4 nucleic acid may be mixed with pharmaceutically acceptable carriers or diluents when used in therapy.

A TRL4 nucleic acid as described herein, such as a TRL4 DNA molecule, may be provided as part of a recombinant vector. For example, a recombinant vector may comprise a TRL4 nucleic acid for use as a template from which TRL4 RNA molecules may be transcribed.

A method of producing a TRL4 nucleic acid as described herein may comprise transcribing a TRL4 nucleotide sequence contained within a recombinant vector.

A recombinant vector may comprise one or more control sequences operably linked to the TRL4 nucleic acid sequence to control its transcription. The term “operably linked” refers to a juxtaposition wherein the components described are in a relationship permitting them to function in their intended manner. A control sequence “operably linked” to a coding sequence is joined in such a way that expression of the TRL4 nucleic acid sequence is achieved under conditions compatible with the control sequences.

Suitable vectors can be chosen or constructed, containing appropriate regulatory sequences, including promoter sequences for driving transcription of the TRL4 nucleic acid template, terminator fragments, polyadenylation sequences, enhancer sequences, marker genes and other sequences as appropriate. Systems for cloning and transcription of nucleic acid in a variety of different host cells are well known. For example, vectors may be plasmids, viral e.g. ‘phage phagemid or baculoviral, cosmids, YACs, BACs, or PACs as appropriate. The vector may also be adapted to be used in vivo, for example for production of TRL4 RNA molecules in situ in neural cells, such as glial cells or neurons. Suitable vectors for in vivo applications include vectors based on adenovirus, adeno-associated virus, retrovirus (such as HIV or MLV), lentivirus or alpha-virus vectors. Suitable vectors are well-known in the art. Vectors may be suitable for administration to an individual to transform or transfect neural cells in vivo in the individual or may be adapted to transform or transfect cells in vitro or ex vivo which may then be administered to the individual.

Vectors may be used in vitro for the production of TRL4 RNA molecules. In some embodiments, a phage RNA polymerase promoter may be operably linked to the inserted TRL4 template and the TRL4 RNA molecule obtained by in vitro transcription. For example, the vector may be incubated with ribonucleotide triphosphates, buffers, magnesium ions, and an appropriate phage RNA polymerase, such as SP6, T7 and T3 polymerase, under conditions for transcription of TRL4 RNA molecules from the coding sequence. Suitable techniques are well-known in the art and appropriate reagents are commercially available (e.g. Applied Biosystems/Ambion, Tex. USA).

Vectors containing TRL4 nucleic acid may be used to transfect or transform a host cell. TRL4 nucleic acid may then be obtained by culturing the host cells so that replication of the vector and/or transcription of TRL4 nucleic acid template contained in the vector occurs. The vector containing TRL4 nucleic acid or transcribed TRL4 nucleic acid may then be recovered from the host cells or the surrounding medium. Prokaryotic and eukaryotic cells are used for this purpose in the art, including strains of E. coli, yeast, and eukaryotic cells such as COS or CHO cells.

Following production, a TRL4 nucleic acid or vector comprising TRL4 nucleic acid may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below). A vector comprising TRL4 nucleic acid may be used to transform or transfect host cells, using standard techniques.

In other embodiments, host cells comprising a vector which comprises a TRL4 nucleic acid may be isolated and/or purified and subsequently used as desired, e.g. in the formulation of a composition which may include one or more additional components, such as a pharmaceutical composition which includes one or more pharmaceutically acceptable excipients, vehicles or carriers (e.g. see below). Suitable host cells include neural cells, such as glial cells or neurons.

TRL4 nucleic acid as described herein may be used in neuroprotective therapy without additional targeting moieties. For example, a TRL4 nucleic acid may be administered directly to neurons undergoing or at risk of cell death or may be expressed in situ in the neurons or in adjacent cells, including implanted cells, from a recombinant vector, as described above. A method of treatment of a neurodegenerative disorder in individual in need thereof may comprise administering a vector which expresses a TRL4 nucleic acid or a cell comprising such a vector to the individual.

In other embodiments, the TRL4 nucleic acid may be linked to a neuronal cell targeting moiety. Neuron targeting moieties are molecules or groups which specifically target neuronal cells. For example, a neuronal targeting moiety may bind specifically to neuronal cells, for example, doperminergic, hippocampal neurons (Lewis et al 1998) or motor neurons (Teng et al 2005), relative to other cell types. Suitable neuron targeting moieties may, for example, bind to the neuronal acetylcholine receptor, neurotrophin receptor (Langevin et al 2002, Tuffereau 2007) or neural cell adhesion molecule (NCAM) (Thoulouze 1998).

In preferred embodiments, neuron targeting moieties also allow passage from the bloodstream into the CNS across the blood-brain barrier. This allows the transvascular delivery of the TRL4 nucleic acid.

Preferred neuron targeting moieties include peptides, such as peptides comprising the amino acid sequence of SEQ ID NO: 3 or a variant thereof.

An amino acid sequence which is a variant of SEQ ID NO: 3 may share at least 80% sequence identity with the SEQ ID NO: 3, preferably at least 90%, at least 95% or at least 98% sequence identity. Sequence identity is described in more detail above.

Particular amino acid sequence variants may differ from the reference nucleotide sequence by addition, deletion or substitution of 1 or more nucleotides, for example, up to 5 amino acid residues.

Suitable neuron targeting peptides are described in more detail in Kumar, P et al 2007.

Neuron targeting peptides may be generated wholly or partly by chemical synthesis. For example, peptides may be synthesised using liquid or solid-phase synthesis methods; in solution; or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Chemical synthesis of peptides is well-known in the art (J. M. Stewart and J. D. Young, Solid Phase Peptide Synthesis, 2nd edition, Pierce Chemical Company, Rockford, Ill. (1984); M. Bodanzsky and A. Bodanzsky, The Practice of Peptide Synthesis, Springer Verlag, New York (1984); J. H. Jones, The Chemical Synthesis of Peptides. Oxford University Press, Oxford 1991; in Applied Biosystems 430A Users Manual, ABI Inc., Foster City, Calif.; G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997).

Alternatively, peptides may be generated wholly or partly by recombinant techniques. For example, a nucleic acid encoding a neuron targeting peptide may be expressed in a host cell and the expressed polypeptide isolated and/or purified from the cell culture.

The mode of attachment of the TRL4 nucleic acid to the neuron targeting moiety will vary depending, in part, on the chemical nature of the targeting moiety. Any convenient method may be employed and suitable conjugation techniques are well-known in the art (see for example, Hermanson, G., ‘Bioconjugate techniques’, Academic Press, San Diego, USA, 1996).

The neuronal targeting moiety may be attached directly to the TRL4 nucleic acid or may be attached indirectly through one or more linker molecules. Attachment may be via one or more covalent bonds or via non-covalent interactions.

In some embodiments, a peptidyl neuron targeting moiety may be linked to the TRL4 nucleic acid by ionic interactions. For example, the neuron targeting peptide may comprise a positively charged region which binds to a negatively charged TRL4 nucleic acid, such as a TRL4 DNA or RNA molecule. Suitable positively charged regions include poly-arginine, preferably poly-D-arginine, for example [L-Arg]₉ or [D-Arg]₉ (Kumar et al 2007). For example, a neuron targeting peptide comprising the sequence of SEQ ID NO: 4 may be employed.

In some preferred embodiments, a complex comprising a TRL4 nucleic acid as described herein, for example a TRL4 nucleic acid comprising the nucleotide sequence of SEQ ID NO: 1 or a variant thereof, linked to a neuron targeting peptide, for example a peptide comprising the sequence of SEQ ID NO: 3 or more preferably SEQ ID NO: 4, may be used in a method of treatment of the human or animal body, for example a method of treating a neurodegenerative disorder, such as Parkinson's disease.

A complex comprising a TRL4 nucleic acid linked to a neuronal cell targeting moiety may be administered to an individual for the treatment of a neurodegenerative disorder, as described herein. As the neuronal cell targeting moiety allows passage across the blood brain barrier, the complex may be administered parenterally into the bloodstream, for example by injection.

Neurodegenerative disorder suitable for treatment as described herein include α-synucleinopathies, such as Parkinson's disease, tauopathies, such as Alzheimer's disease, codon reiteration mutation disorders such as Huntington's disease and other neurodegenerative conditions such as Amyotrophic Lateral Sclerosis (ALS).

In some preferred embodiments, the neurodegenerative disorder is Parkinson's disease.

Other conditions suitable for treatment as described herein include disorders mediated by cell death, such as chronic rejection and ischemia/reperfusion disease.

In some embodiments, the neurodegenerative disorder or disorder mediated by cell death may be characterised by mitochondrial dysfunction, oxidative stress and/or abnormalities in mitochondrial complex I in neuronal cells.

The term “treatment” in the context of treating a neurodegenerative disorder, pertains generally to treatment and therapy, whether of a human or an animal (e.g. in veterinary applications), in which a desired therapeutic effect is achieved, for example, the inhibition of the progress of the disorder, and includes a reduction in the rate of progress, a halt in the rate of progress, amelioration of the disorder, and cure of the disorder. Treatment as a prophylactic measure (i.e. prophylaxis) is also included.

While it is possible for an active compound such as an TRL4 nucleic acid; a vector or cell expressing a TRL4 nucleic acid; or an complex comprising a TRL4 nucleic acid linked to a neuronal targeting peptide as described above, to be administered alone, it is preferable to present it as a pharmaceutical composition (e.g. formulation) comprising the TRL4 nucleic acid, vector, cell, or complex, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Pharmaceutical compositions comprising a TRL4 nucleic acid, vector, cell, or complex, as described above, for example, admixed or formulated together with one or more pharmaceutically acceptable carriers, excipients, buffers, adjuvants, stabilisers, or other materials, as described herein, may be used in the methods described herein.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well-known in the art of pharmacy.

Such methods include the step of bringing the active compound into association with a carrier which may constitute one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The TRL4 nucleic acid, vector, cell, or complex or pharmaceutical composition comprising the TRL4 nucleic acid, vector, cell, or complex may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); parenteral, for example, by injection, including subcutaneous, intradermal, intracranial, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Formulations suitable for parenteral administration include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example, from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use.

Examples of techniques and protocols mentioned above can be found in Remington's Pharmaceutical Sciences, 16th edition, Osol, A. (ed), 1980.

It will be appreciated that appropriate dosages of the TRL4 nucleic acid, vector, cell, or complex and compositions comprising the TRL4 nucleic acid, vector, cell, or complex can vary from patient to patient. Determining the optimal dosage will generally involve the balancing of the level of diagnostic benefit against any risk or deleterious side effects of the administration. The selected dosage level will depend on a variety of factors including, but not limited to, the route of administration, the time of administration, the rate of excretion of the TRL4 nucleic acid, vector, cell, or complex, other drugs, compounds, and/or materials used in combination, and the age, sex, weight, condition, general health, and prior medical history of the patient. The amount of TRL4 nucleic acid and route of administration will ultimately be at the discretion of the physician, although generally the dosage will be to achieve concentrations of the TRL4 nucleic acid at a lesion site without causing substantial harmful or deleterious side-effects.

Administration in vivo can be effected in one dose, continuously or intermittently (e.g., in divided doses at appropriate intervals). Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the physician.

The composition may be administered in a localised manner to a desired site or may be delivered in a manner in which it targets particular cells or tissues. For example, the composition may be administered directly to neuronal tissue.

A composition may be administered alone or in combination with other treatments, either simultaneously or sequentially dependent upon the condition to be treated.

Various further aspects and embodiments of the present invention will be apparent to those skilled in the art in view of the present disclosure. All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXPERIMENTS

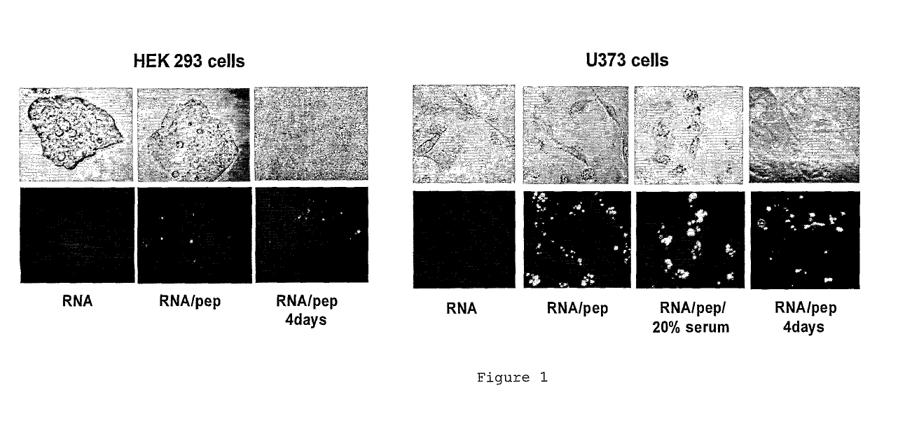

Non-neuronal HEK293 cells or neuronal U373 cells were treated with in vitro transcribed TRL4 RNA, labelled with FITC-conjugated dCTP, which had been complexed with RVG peptide at a molar ratio of 10:1. Cells were analysed 1 day post-addition (RNA/pep) or 4 days post-addition (RNA/pep 4 days). For the U373 analysis, RNA/peptide complex was also incubated at 37° C. for 2 h in 20% serum prior to addition to cells. FIG. 1 (upper panels) show phase contrast photomicrographs and FIG. 1 (lower panels) show FITC staining to detect the labelled RNA/pep complex. These results show that RVG peptide is able to deliver a fluorescently-labelled TRL4 RNA specifically to U373 neuronal cells (not non-neuronal HEK293 cells) where it is stable for up to 4 days on the treated cells and is also stable after incubation for 4 hours in high concentrations of serum prior to addition to the cells.

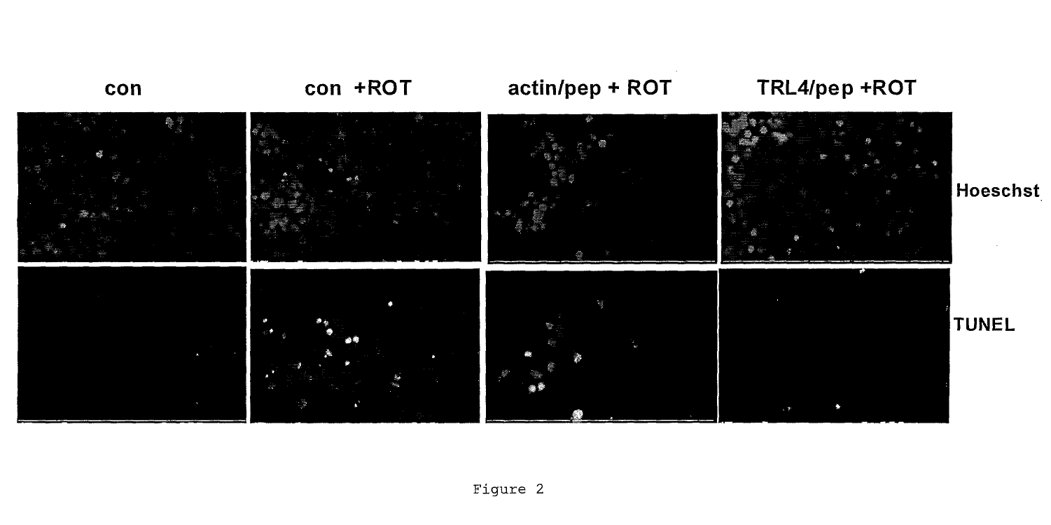

Next, SH-SY5Y cells were left untreated (C) or treated with an RNA/peptide complex containing in vitro transcribed actin RNA (actin/pep) or in vitro transcribed TRL4 RNA (TRL4/pep). 48 hours later cells were treated with rotenone and 18 hours after that, assayed for cell death by TUNEL fluorescence. FIG. 2 shows TUNEL fluorescence in red and total cell numbers identified by staining of nuclei with Hoechst. These results show that the RNA/peptide complex specifically protects dopaminergic cells from cell death in an accepted cell culture model of PD-rotenone-induced death of SH-SY5Y cells.

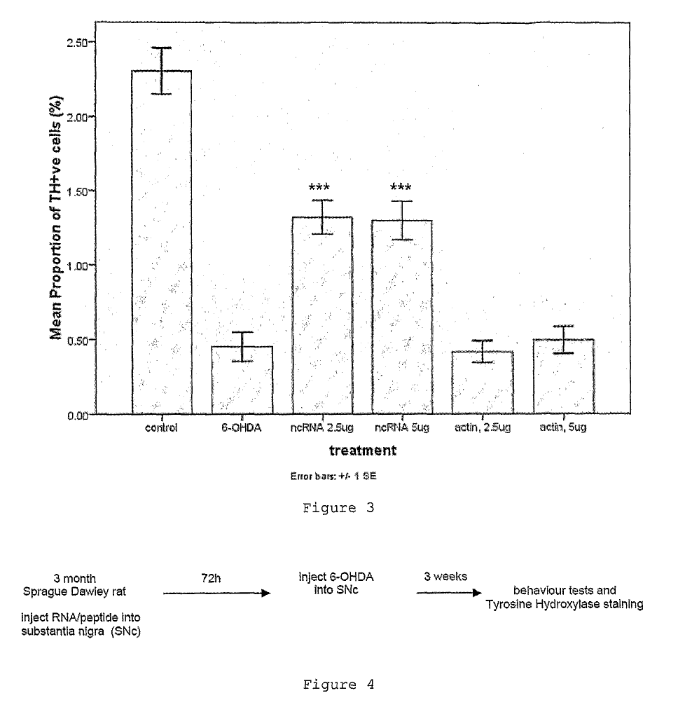

To test the activity of the RNA/peptide complex in primary rat brain explants, rat 13.5 day foetal ventral midbrain primary cultures were left untreated (control), treated for 24 h with actin complexed with RVG peptide (actin/peptide) or treated for 24 h with TRL4 RNA complexed with RVG peptide (TRL4/peptide). Cultures were then subjected to 60 μM 6-OHDA for 48 hours. Cells were then stained for tyrosine hydroxylase (TH) and the cultures scored for relative proportions of TH positive cells in TRL4 treated (ncRNA) or control actin (actin) RNA explants. The results are shown in FIG. 3. Post-hoc Bonferroni analysis revealed that cells receiving TRL4/peptide (“ncRNA”) treatment (2.5 ug or 5 ug) and 6-OHDA have fewer TH-positive cells than the control cells not treated with 6-OHDA. However, cells receiving TRL4/peptide (“ncRNA”) treatment and 6-OHDA showed a significantly higher proportion of dopaminergic neurons compared to cells treated with 6-OHDA only, or to cells treated with the control actin complex and 6-OHDA. These results show that the viral RNA, but not the actin control RNA, prevents 6-OHDA-induced death of primary rat brain explants.

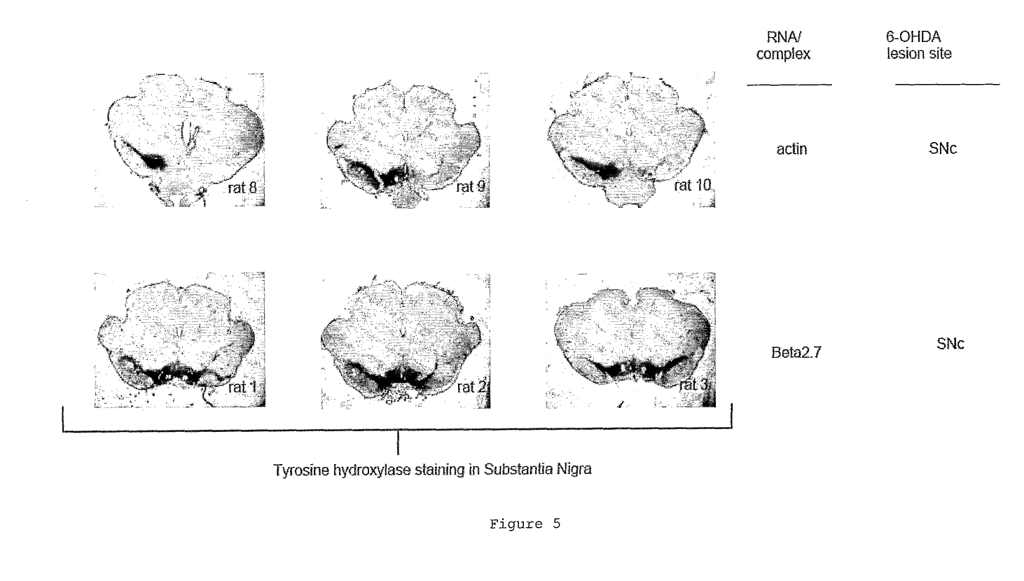

We next tested whether the TRL4 complexed with RVG peptide showed any neuroprotection in a unilateral 6-OHDA rat lesion model of Parkinson's disease. As shown in FIG. 4, the protective peptide/RNA was directly injected into the substantia nigra and assessed using TH staining of brain sections. The TRL4/peptide treated rats but not control actin/peptide treated rats were found to be protected from the 6-OHDA lesion (FIG. 5). Robust TH staining was detected in the substantia nigra (FIG. 5) as well as the striatum.

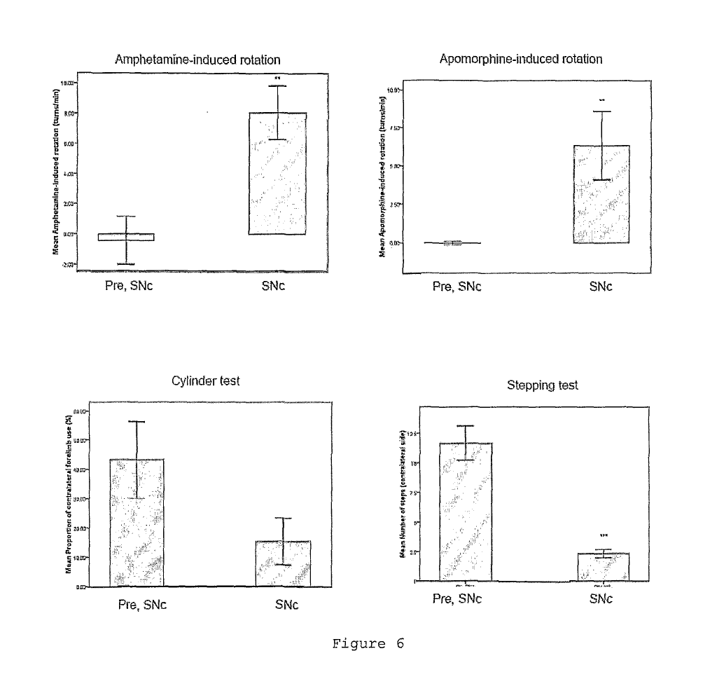

To confirm these results, behavioural studies were performed on 6-OHDA lesioned rats pre-treated with TRL4/peptide (pre,SNc) and 6-OHDA lesioned rats with actin/peptide pre-treatment (SNc). The results of these behavioural studies are shown in FIG. 6. Pretreatment with TRL4/peptide is shown to confer neuroprotection on 6-OHDA lesioned rats pre-treated (pre,SNc) compared to actin/peptide pre-treatment (SNc) of 6-OHDA lesioned rats.

Both immunohistochemistry and behavioural studies therefore show that TRL4/peptide pretreatment confers neuroprotection against intranigral lesion with 6-OHDA, compared to rats receiving control actin/peptide pretreatment, and ameliorates both lesion-induced behavioural and biochemical deficits.

### REFERENCES

Dowd, E et al Brain Res Bull 2005; 68:24.
Kuan, W L et al Neurobiol Dis 2007; 25:594.
Kumar, P et al Nature 2007; 448:39-43
Langevin, C. et al. (2002) J Biol Chem 277(40): 37655-62.
Langevin, C. et al. (2002) J Virol 76(21): 10756-65.
McSharry B et al Journal of General Virology (2003), 84, 2511-2516
Michell A W et al Cell Transplant. 2007; 16(5):461-74.
Przedborski, S et al J Bioenerg Biomembr 2004; 36:375.
Pyle, A et al Ann Neurol 2005; 57:564.
Reeves, M B et al Science 2007; 316:1345
Schapira, A H et al J Neurochem 1990; 54:823.
Teng, Q. et al. (2005). Neurobiol Dis 20(3): 694-700.
Thoulouze, M. I., M. Lafage, et al. (1998). J Virol 72(9): 7181-90.
Tofaris G K et al J Neuroscience 26, 3942-3950, 2006.
Tuffereau, C., K. Schmidt, et al. (2007). J Virol 81(24): 13622-30.
Watabe, M. and T. Nakaki (2004) J Pharmacol Exp Ther 311(3): 948-53.

### Sequences

| | |
|---|---|
| 1 | ccagatcgct gctgccccgg cgttctccag aagccccggc gggcgaatcg gccggctggt |
| 61 | cggtcggcgc tcggacggat ggggagaacg gcggtgactt agccgcccgt ggccgggaga |
| 121 | agacggggga gccgagatga caacagcagt cgtggaaggg tcgccaagcc ccggtccttc |
| 181 | tcttctgtct ggtcgaatct cgttttcttt tttcaaccgc tctttttgtc acctttttat |
| 241 | gtgagtttct cttccgcgtc tcccggccgt accatccacc catgcagcat gcacgcgtgt |
| 301 | atgtatgcat cgcctctcct ccgtcccgac taccatcagc agtaccactg ccgccacccc |
| 361 | cagcgccacc accgctgccg tcgccaccgc gttatccatt cctcagggc tggtcctggg |
| 421 | gaacgggtcg gcggccggtc ggcttctgtt ttattatttt ttttttattt tttatcttct |
| 481 | cctttcctta atctcggatt atcatttccc tctcctacct accacgaatc gcagatgata |
| 541 | aacaagaggg taaaaagaaa aaagctacag acatttgggt acctcagctt tccgataact |
| 601 | cgaagaattc aaagtcgacg attcccaaca agagaaaaca gaacaaaaac aaggtcattt |
| 661 | ttatttatcc tcatcgtcaa caacaactac cgacaacaac gaaacaccac caagaatgtc |
| 721 | aatccgcaag ggtgttcctg ccccctcgac gcgcctgtcg cgatcctcat ggcgaggacc |
| 781 | gcgatctccg tatag |

SEQ ID NO: 1 TRL4 nucleotide sequence (residues 1 to 795 of SEQ ID NO: 2)

| | |
|---|---|
| 1 | ccagatcgct gctgccccgg cgttctccag aagccccggc gggcgaatcg gccggctggt |
| 61 | cggtcggcgc tcggacggat ggggagaacg gcggtgactt agccgcccgt ggccgggaga |
| 121 | agacggggga gccgagatga caacagcagt cgtggaaggg tcgccaagcc ccggtccttc |
| 181 | tcttctgtct ggtcgaatct cgttttcttt tttcaaccgc tctttttgtc acctttttat |
| 241 | gtgagtttct cttccgcgtc tcccggccgt accatccacc catgcagcat gcacgcgtgt |
| 301 | atgtatgcat cgcctctcct ccgtcccgac taccatcagc agtaccactg ccgccacccc |
| 361 | cagcgccacc accgctgccg tcgccaccgc gttatccatt cctcgtaggc tggtcctggg |
| 421 | gaacgggtcg gcggccggtc ggcttctgtt ttattatttt ttttttattt tttatcttct |
| 481 | cctttcctta atctcggatt atcatttccc tctcctacct accacgaatc gcagatgata |
| 541 | aacaagaggg taaaaagaaa aaagctacag acatttgggt acctcagctt tccgataact |
| 601 | cgaagaattc aaagtcgacg attcccaaca agagaaaaca gaacaaaaac aaggtcattt |
| 661 | ttatttatcc tcatcgtcaa caacaactac cgacaacaac gaaacaccac caagaatgtc |
| 721 | aatccgcaag ggtgttcctg ccccctcgac gcgcctgtcg cgatcctcat ggcgaggacc |
| 781 | gcgatctccg tataggtaga tgaaattatc ccgtgtccgg tcctgattcc ccgcatgccc |
| 841 | tgcacatcct gacgcgtcgg tcagcagcca aacaatcata ggaaatgaac cagaagaaca |
| 901 | aaaagatcat ctctctcggt gtatagcaac accaacaaca accgcatcgc aacatcttca |
| 961 | tccgcaagac ggaaagaaaa caacaataat gagaatgaaa tcaccacaac caagccagat |
| 1021 | ttcacgtcca tgagttttta ttatattatt atcaaaacga aaaacagaaa aactgtcata |
| 1081 | gataaatata aaaaaaatag aaaccacaaa cgactactag tactccaatc ttagatgtat |
| 1141 | atgctcctag ataagattta gtattaccat aatcatcgaa gaatgaaaga cgacgatgat |
| 1201 | tccttaccgt cctgccaccc ggtctgtatg tagagagaga agagagaaaa cggtgaatcc |
| 1261 | aagatccccg ggtcggcgtc ggcatgccgc tgatcgcagt ggccccacct cggcatgccg |
| 1321 | gcgccgggcg aggaattgct catgaaaaaa gtatctttct gtaaaaaaag aaaacaatac |
| 1381 | atgattaacc gaaaagaaac caacaaaaag aacccgagat cagtcgattt cgatcactac |
| 1441 | gataaacaca tggaagattt cttgaaaaaa gaaaagagaa agagaccacc ttcccggcgg |
| 1501 | cggacacgct cctctccgtc gccgttctgc accatgattc gatcaataac aacatcatca |
| 1561 | tcggagacca tcttttaatc aatcagcgtt gcagtagtcg actccctgga cacgaaggag |
| 1621 | tcatccattt ttatcctcgc acttcttcgc tctcaaagcc gcctttaaag ttgaaatgaa |
| 1681 | aggatggaaa catggaatac agttttaatt gcacgtatca ccattttact acaaaaagaa |
| 1741 | aaaaaaacaa cttacacata gtattacctt aggtttacgg ataagtagag tgtaggcgtt |
| 1801 | tttgaaacag ttcagccaat gcaatcttgt ctcggcataa tcactctttc tgcatataat |
| 1861 | agtagtagta gatttattca catcaacaca gcgaaaaact ccagcatcaa agtacaccta |
| 1921 | gagacagccc ttaaaatata gtttgcagct tttagatgta cttacaccaa agaagattac |
| 1981 | cgtccttacg agaaaacaga tactcggata taggaatcaa ggcagctctg cactgaaaac |
| 2041 | acactctcct gtcacgacac cgcgccacac cagaggcgta cgcgtgactt catcgcaacg |
| 2101 | atccatcgtg atgtccctcg cagaacctaa aaagaccaaa aaaaaatctt ggaccacagt |
| 2161 | tgtcgattct tgaagacaat attctcgtga gaactttgag attcgcactt gaaacctctt |
| 2221 | aggatccaca aaaacaacaa cctctgtatg gaaaatgcgc tattttatct cagcttttct |
| 2281 | cccaaacctc ggtttcttcc tattcttaag ttttccctag tatatttgcc tccttataag |
| 2341 | aaaagaagca caagctcggt cgcacggatt attccttctg ctaatctatt attttgttcc |
| 2401 | tttttttttt ctttgccttc |

SEQ ID NO: 2 beta2.7 nucleotide sequence (NCBI AY325309.1 GI: 33114657)

YTIWMPENPRPGTPCDIFTNSRGKRASNG

SEQ ID NO: 3 RVG amino acid sequence

YTIWMPENPRPGTPCDIFTNSRGKRASNGGGGRRRRRRRRR

SEQ ID NO: 4 RVG9R (9 residue D-Arg tail)

| | |
|---|---|
| 1 | ccagatcgct gctgctccgg cgttctccag aagccccggc gggcgaatcg gccggctggt |
| 61 | cggtcggcgc tcggacggat ggggagaacg gcggtgactt agccgcccgt ggccgggaga |
| 121 | agacggagga gccgagatga caacagcagt cgtggaaggg tcgccaagcc ccggtccttc |
| 181 | tcttctgtct ggtcgaatct cgttttcttt tttcaaccgc tctttttgtc acctttttat |
| 241 | gtgagtttct cttccgcgtc tcccggtcgg accatccacc catgcagcat gcacgcgtgt |
| 301 | atgtatgcat cgtctctcct ccgtcccgac taccatcagc agtaccacta ccgccacccc |
| 361 | cagcgccacc accgctgccg tcgccaccgc gttatccgtt cctcgtaggc tggtcctggg |
| 421 | gaacgggtcg gcggccggtc ggcttctgtt ttattttttt ttttattttt tatcttctcc |
| 481 | tttccttaat ctcggattat catttccctc tcctacctac cacgaatcgc agatgataaa |
| 541 | caagagggta aaaagaaaaa agctacagac atttgggtac ctcagctttc cgataactcg |
| 601 | aagaattcaa agtcgacgat tcccaacaag agaaaacaga acaaaaacaa ggtcattttt |
| 661 | atttatcctc atcgtcaaca acaactaccg acaacaacga aataccacca agaatgtcaa |
| 721 | tccgcaaggg tgttcctgcc ccctcgacgc gcctgtcgcg atcctcatgg cgaggaccgc |
| 781 | gatctccgta tag |

SEQ ID NO: 5 TRL4 nucleotide sequence from HCMV Merlin isolate (nucleotides 1 to 793 of AY325310.1 GI: 33114658)

| | |
|---|---|
| 1 | ccagatcgct gctgccccgg cgttctccag aagccccggc gggcgaatcg gccggctggt |
| 61 | cggtcggcgc tcggacggat ggggagaacg gcggtgactt agccgcccgt ggccgggaga |
| 121 | agacggagga gccgagatga caacagcagt cgtggaaggg tcgccaagcc ccggtccttc |
| 181 | tcttctgtct ggtcgaatct cgttttcttt tttcaaccgc tctttttgtc acctttttat |
| 241 | gtgagtttct cttccgcgtc tcccggccgt accatccacc catgcagcat gcacgcgtgt |
| 301 | atgtatgcat gcatcgtctc tcctccgtcc cgactaccat cagcagcacc actaccgcca |
| 361 | cccccagcgc caccaccgct gccgtcgcca ccgcgttatc cgttcctcgt aggctggtcc |
| 421 | tggggaacgg gtcggcggcc ggtcggcttc tgttttatta tttttttttt attttttatc |
| 481 | ttctcctttc cttaatctcg gattatcatt tccctctcct acctaccacg aatcgcagat |
| 541 | gataaacaag agggtaaaaa gaaaaaagct acagacattt gggtacctca gctttccgat |
| 601 | aactcgaaga attcaaagtc gacgattccc aacaaaagaa aacagaacaa aaacaaggtc |
| 661 | atttttattt atcctcatcg tcaacaacaa ctaccgacaa caacgaaaca ccaccaagaa |
| 721 | tgtcaatccg caagggtgtt cctgccccct cgacgcgcct gtcgcgatcc tcatggcgag |
| 781 | gaccgcgatc tccgtatag |

SEQ ID NO: 6 TRL4 nucleotide sequence from HCMV 3157 isolate (nucleotides 1 to 799 of AY325311.1 GI: 33114659)

| | |
|---|---|
| 1 | ccagatcgct gctgccccgg cgttctccag aagccccggc gggcgaatcg gccggctggt |
| 61 | cggtcggcgc tcggacggat ggggagaacg gcggtgactt agccggccgt gtccgggaga |
| 121 | agacggagga gccgagatga caacagcagt cgtggaaggg tcgccaagcc ccggcccttc |
| 181 | tcttctgtct ggtcgaatct cgttttcttt tttcaaccgc tctttttgtc acctttttat |
| 241 | gtgagtttct cttccgcgtc tcccggccgt accatccacc catgcagcat gcacgcgtgt |
| 301 | atgtatgcat cgtctctcct ccgtcccgac taccatcagc agcaccacta ccgccacccc |
| 361 | cagcgccacc accgctgccg tcgccaccgc gttatccgtt cctcgtaggc tggtcctggg |
| 421 | gaacgggtcg gcggccggtc ggcttctgtt ttattatttt ttttttattt tttatcttct |
| 481 | cctttcctta atctcggatt atcatttccc tctcctacct accacgaatc gcagatgata |
| 541 | aacaagaggg taaaaagaaa aaagctacag acatttgggt acctcagctt tccgataact |
| 601 | cgaagaattc aaagtcgacg attcccaaca agagaaaaca gaacaaaaac aaggtcattt |
| 661 | ttatttatcc tcatcgtcaa caacaactac cgacaacaac gaaacaccac caagaatgtc |
| 721 | aatccgcaag ggtgttcctg ccccctcgac gcgcctgtcg cgatcctcat ggcgaggacc |
| 781 | gcgatctccg tatag |

SEQ ID NO: 7 TRL4 nucleotide sequence from HCMV 6397 isolate (nucleotides 1 to 795 of AY325312.1 GI: 33114660)

| | |
|---|---|
| 1 | CCAGATCGCT GCTGCCCCGG CGTTCTCCAG AAGCCCCGGC GGGCGAATCG |
| 51 | GCCGGCTGGT CGGTCGGCGC TCGGACGGAT GGGGAGAACG GCGGTGACTT |
| 101 | AGCCGCCCGT GGCCGGGAGA AGACGGAGGA GCCGAGATGA CAACAGCAGT |
| 151 | CGTGGAAGGG TCGCCAAGCC CCGGTCCTTC TCTTCTGTCT GGTCGAATCT |
| 201 | TGTTTTCTTT TTTCAACCGC TCTTTTTGTC ACCTTTTTAT GTGAGTTTCT |
| 251 | CTTCCGCGTC TCCCGGCCGT ACCATCCACC CATGCAGCAT GCACGCGTGT |
| 301 | ATGTATGCAT CGCCTCTCCT CCGTCCCGAC TACCATCAGC AGTACCACTG |
| 351 | CCGCCACCCC CAGCGCCACC ACCGCTGCCG TCGCCACCGC GTTATCCGTT |
| 401 | CCTCGTAGGC TGGTCCTGGG GAACGGGTCG GCGGCCGGTC GGCTTCTGTT |
| 451 | TTATTATTTT TTTTTATTTT TTATCTTCTC CTTTCCTTAA TCTCGGATTA |
| 501 | TCATTTCCCT CTCCTACCTA CCACGAATCG CAGATGATAA ACAAGAGGGT |
| 551 | AAAAAGAAAA AAGCTACAGA CATTTGGGTA CCTCAGCTTT CCGATAACTC |
| 601 | GAAGAATTCA AAGTCGACGA TTCCCAACAA GAGAAAACAG AACAAAAACA |
| 651 | AGGTCATTTT TATTTATCCT CATCGTCAAC AACAACTACC GACAACAACG |
| 701 | AAACACCACC AAGAATGTCA ATCCGCAAGG GTGTTCCTGC CCCCTCGACG |
| 751 | CGCCTGTCGC GATCCTCATG GCGAGGACCG CGATCTCCGT ATAG |

SEQ ID NO: 8 TRL4 nucleotide sequence from AD169 isolate (reverse complement of nucleotides 3782 to 4575 of X17403.1 GI:59591)

1. An isolated TRL4 nucleic acid consisting of a nucleotide sequence having at least 60% sequence identity to SEQ ID NO: 1.

2. A TRL4 nucleic acid according to claim 1 wherein the TRL4 nucleic acid consists of the nucleotide sequence of SEQ ID NO: 1, SEQ ID NO: 5, SEQ ID NO: 6, SEQ ID NO: 7 or SEQ ID NO: 8.

3. A TRL4 nucleic acid according to claim 1 which is an RNA molecule.

4. A vector comprising a TRL4 nucleic acid according to claim 1.

5. A host cell comprising a TRL4 nucleic acid according to claim 1.

6. A complex comprising a TRL4 nucleic acid which comprises a nucleotide sequence having at least 60% sequence identity to SEQ ID NO: 1,
said TRL4 nucleic acid being linked to a neuronal cell targeting moiety.

7. A complex according to claim 6 wherein the TRL4 nucleic acid comprises the nucleotide sequence of SEQ ID NO: 1, SEQ ID NO: 2, SEQ ID NO: 5, SEQ ID NO: 6, SEQ ID NO: 7 or SEQ ID NO: 8.

8. (canceled)

9. A complex according to claim 6 wherein the neuron targeting moiety is a peptide comprising an amino acid sequence having at least 80% sequence identity to SEQ ID NO: 3.

10. A complex according to claim 7, wherein the peptide comprises the amino acid sequence of SEQ ID NO: 3.

11. A complex according to claim 9, wherein the neuron targeting peptide comprises a polyarginine sequence which binds the TRL4 nucleic acid through ionic interactions.

12. A complex according to claim 11 wherein the peptide comprises the amino acid sequence of SEQ ID NO: 4.

13. (canceled)

14. A pharmaceutical composition comprising a pharmaceutically acceptable carrier and;
a TRL4 nucleic acid comprising a nucleotide sequence having at least 60% sequence identity to SEQ ID NO: 1;
a recombinant vector comprising a TRL4 nucleotide sequence having at least 60% sequence identity to SEQ ID NO: 1;
a host cell comprising a heterologous TRL4 nucleotide sequence having at least 60% sequence identity to SEQ ID NO: 1; or,
a complex comprising a TRL4 nucleic acid which comprises a nucleotide sequence having at least 60% sequence identity to SEQ ID NO: 1, said TRL4 nucleic acid being linked to a neuronal cell targeting moiety.

15.-18. (canceled)

19. A method of treatment of a neurodegenerative disorder in individual in need thereof comprising;
administering to the individual one of:
a TRL4 nucleic acid comprising a nucleotide sequence having at least 60% sequence identity to SEQ ID NO: 1;
a recombinant vector comprising a TRL4 nucleotide sequence having at least 60% sequence identity to SEQ ID NO: 1;
a host cell comprising a heterologous TRL4 nucleotide sequence having at least 60% sequence identity to SEQ ID NO: 1; or
a complex comprising a TRL4 nucleic acid which comprises a nucleotide sequence having at least 60% sequence identity to SEQ ID NO: 1, said TRL4 nucleic acid being linked to a neuronal cell targeting moiety.

20. (canceled)

21. The method according to claim 19 wherein the TRL4 nucleic acid comprises the nucleotide sequence of SEQ ID NO: 1, SEQ ID NO:2, SEQ ID NO: 5, SEQ ID NO: 6, SEQ ID NO: 7 or SEQ ID NO: 8.

22. The method according to claim 21, wherein the TLR4 nucleic acid comprises the nucleotide sequence of SEQ ID NO: 2.

23. The method according to claim 19, wherein the neurodegenerative disorder is selected from the group consisting of codon reiteration mutation disorders, α-synucleinopathies and tauopathies.

24. The method according to claim 23, wherein the neurodegenerative condition is selected from the group consisting of Parkinson's Disease, Alzheimer's Disease, Huntington's Disease and Amyotrophic Lateral Sclerosis.

25. A method of producing a TRL4 nucleic acid comprising transcribing a TRL4 nucleic acid according to claim 1 which is contained within a recombinant vector to produce transcribed TRL4 nucleic acid.

26. A method according to claim 25 comprising one or both of isolating or purifying the transcribed TRL4 nucleic acid.


SEQUENCE LISTING (8 sequences)

SEQ ID NO: 1 - DNA, 794 bp, Human cytomegalovirus
ccagatcgctgctgccccggcgttctccagaagccccggcgggcgaatcggccggctggtcggtcggcgctcggacggatggggagaacggcggtgacttagccgcccgtggccgggagaagacgggggagccgagatgacaacagcagtcgtggaagggtcgccaagccccggtccttctcttctgtctggtcgaatctcgttttcttttttcaaccgctctttttgtcacctttttatgtgagtttctcttccgcgtctcccggccgtaccatccacccatgcagcatgcacgcgtgtatgtatgcatcgcctctcctccgtcccgactaccatcagcagtaccactgccgccacccccagcgccaccaccgctgccgtcgccaccgcgttatccattcctcagggctggtcctggggaacgggtcggcggccggtcggcttctgttttattattttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtatag

SEQ ID NO: 2 - DNA, 2420 bp, Human cytomegalovirus
ccagatcgctgctgccccggcgttctccagaagccccggcgggcgaatcggccggctggtcggtcggcgctcggacggatggggagaacggcggtgacttagccgcccgtggccgggagaagacgggggagccgagatgacaacagcagtcgtggaagggtcgccaagccccggtccttctcttctgtctggtcgaatctcgttttcttttttcaaccgctctttttgtcacctttttatgtgagtttctcttccgcgtctcccggccgtaccatccacccatgcagcatgcacgcgtgtatgtatgcatcgcctctcctccgtcccgactaccatcagcagtaccactgccgccacccccagcgccaccaccgctgccgtcgccaccgcgttatccattcctcgtaggctggtcctggggaacgggtcggcggccggtcggcttctgttttattattttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtataggtagatgaaattatcccgtgtccggtcctgattccccgcatgccctgcacatcctgacgcgtcggtcagcagccaaacaatcataggaaatgaaccagaagaacaaaaagatcatctctctcggtgtatagcaacaccaacaacaaccgcatcgcaacatcttcatccgcaagacggaaagaaaacaacaataatgagaatgaaatcaccacaaccaagccagatttcacgtccatgagtttttattatattattatcaaaacgaaaaacagaaaaactgtcatagataaatataaaaaaaatagaaaccacaaacgactactagtactccaatcttagatgtatatgctcctagataagatttagtattaccataatcatcgaagaatgaaagacgacgatgattccttaccgtcctgccacccggtctgtatgtagagagagaagagagaaaacggtgaatccaagatccccgggtcggcgtcggcatgccgctgatcgcagtggccccacctcggcatgccggcgccgggcgaggaattgctcatgaaaaaagtatctttctgtaaaaaaagaaaacaatacatgattaaccgaaaagaaaccaacaaaaagaacccgagatcagtcgatttcgatcactacgataaacacatggaagatttcttgaaaaaagaaaagagaaagagaccaccttcccggcggcggacacgctcctctccgtcgccgttctgcaccatgattcgatcaataacaacatcatcatcggagaccatcttttaatcaatcagcgttgcagtagtcgactccctggacacgaaggagtcatccatttttatcctcgcacttcttcgctctcaaagccgcctttaaagttgaaatgaaaggatggaaacatggaatacagttttaattgcacgtatcaccattttactacaaaaagaaaaaaaaacaacttacacatagtattaccttaggtttacggataagtagagtgtaggcgtttttgaaacagttcagccaatgcaatcttgtctcggcataatcactctttctgcatataatagtagtagtagatttattcacatcaacacagcgaaaaactccagcatcaaagtacacctagagacagcccttaaaatatagtttgcagcttttagatgtacttacaccaaagaagattaccgtccttacgagaaaacagatactcggatataggaatcaaggcagctctgcactgaaaacacactctcctgtcacgacaccgcgccacaccagaggcgtacgcgtgacttcatcgcaacgatccatcgtgatgtccctcgcagaacctaaaaagaccaaaaaaaaatcttggaccacagttgtcgattcttgaagacaatattctcgtgagaactttgagattcgcacttgaaacctcttaggatccacaaaaacaacaacctctgtatggaaaatgcgctattttatctcagcttttctcccaaacctcggtttcttcctattcttaagttttccctagtatatttgcctccttataagaaaagaagcacaagctcggtcgcacggattattccttctgctaatctattattttgttccttttttttttctttgccttc

SEQ ID NO: 3 - protein, 29 aa, Rabies virus
YTIWMPENPRPGTPCDIFTNSRGKRASNG

SEQ ID NO: 4 - protein, 41 aa, Artificial sequence
  Synthetic sequence RVG9R peptide (9 residue D-Arg tail)
YTIWMPENPRPGTPCDIFTNSRGKRASNGGGGRRRRRRRRR

SEQ ID NO: 5 - DNA, 793 bp, Human cytomegalovirus
ccagatcgctgctgctccggcgttctccagaagccccggcgggcgaatcggccggctggtcggtcggcgctcggacggatggggagaacggcggtgacttagccgcccgtggccgggagaagacggaggagccgagatgacaacagcagtcgtggaagggtcgccaagccccggtccttctcttctgtctggtcgaatctcgttttcttttttcaaccgctctttttgtcacctttttatgtgagtttctcttccgcgtctcccggtcggaccatccacccatgcagcatgcacgcgtgtatgtatgcatcgtctctcctccgtcccgactaccatcagcagtaccactaccgccacccccagcgccaccaccgctgccgtcgccaccgcgttatccgttcctcgtaggctggtcctggggaacgggtcggcggccggtcggcttctgttttatttttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaataccaccaagaatgtcaatccgcaagggtgttcctgccccctcgacgcgcctgtcgcgatcctcatggcgaggaccgcgatctccgtatag

SEQ ID NO: 6 - DNA, 799 bp, Human cytomegalovirus
ccagatcgctgctgccccggcgttctccagaagccccggcgggcgaatcggccggctggtcggtcggcgctcggacggatggggagaacggcggtgacttagccgcccgtggccgggagaagacggaggagccgagatgacaacagcagtcgtggaagggtcgccaagccccggtccttctcttctgtctggtcgaatctcgttttcttttttcaaccgctctttttgtcacctttttatgtgagtttctcttccgcgtctcccggccgtaccatccacccatgcagcatgcacgcgtgtatgtatgcatgcatcgtctctcctccgtcccgactaccatcagcagcaccactaccgccacccccagcgccaccaccgctgccgtcgccaccgcgttatccgttcctcgtaggctggtcctggggaacgggtcggcggccggtcggcttctgttttattattttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaaaagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtatag

SEQ ID NO: 7 - DNA, 795 bp, Human cytomegalovirus
ccagatcgctgctgccccggcgttctccagaagccccggcgggcgaatcggccggctggtcggtcggcgctcggacggatggggagaacggcggtgacttagccggccgtgtccgggagaagacggaggagccgagatgacaacagcagtcgtggaagggtcgccaagccccggcccttctcttctgtctggtcgaatctcgttttcttttttcaaccgctctttttgtcacctttttatgtgagtttctcttccgcgtctcccggccgtaccatccacccatgcagcatgcacgcgtgtatgtatgcatcgtctctcctccgtcccgactaccatcagcagcaccactaccgccacccccagcgccaccaccgctgccgtcgccaccgcgttatccgttcctcgtaggctggtcctggggaacgggtcggcggccggtcggcttctgttttattattttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtatag

SEQ ID NO: 8 - DNA, 794 bp, Human cytomegalovirus
ccagatcgctgctgccccggcgttctccagaagccccggcgggcgaatcggccggctggtcggtcggcgctcggacggatggggagaacggcggtgacttagccgcccgtggccgggagaagacggaggagccgagatgacaacagcagtcgtggaagggtcgccaagccccggtccttctcttctgtctggtcgaatcttgttttcttttttcaaccgctctttttgtcacctttttatgtgagtttctcttccgcgtctcccggccgtaccatccacccatgcagcatgcacgcgtgtatgtatgcatcgcctctcctccgtcccgactaccatcagcagtaccactgccgccacccccagcgccaccaccgctgccgtcgccaccgcgttatccgttcctcgtaggctggtcctggggaacgggtcggcggccggtcggcttctgttttattatttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtatag